(19) United States
(12) Patent Application Publication
(10) Pub. No.: US 2006/0015055 A1
(43) Pub. Date: Jan. 19, 2006

(54) Air permeable tape

(76) Inventor: Hai Feng Kuo, Keelung (TW)

Correspondence Address: BIRCH STEWART KOLASCH & BIRCH, PO BOX 747, FALLS CHURCH, VA, 22040-0747, US

(21) Appl. No.: 11/014,957

(22) Filed: Dec. 20, 2004

(30) Foreign Application Priority Data
Jul. 14, 2004 (TW) 093211124

(51) Int. Cl. A61F 13/00; A61F 15/00

(52) U.S. Cl. 602/54; 602/41; 602/45

(57) ABSTRACT

An air permeable tape for adhering on human skin. The air permeable tape includes a plurality of air permeable holes for dissipating the water vapor generated from human skin to the air. An adhesive is formed on the fiber surface of the air permeable tape, thereby securely affixing the air permeable tape onto human skin. The air permeable tape can also be easily tore down.

### BACKGROUND OF THE INVENTION

1. Field of the Invention

The present invention relates generally to an air permeable tape, and more particularly to an air permeable tape that is used in medical treatments.

2. Related Art

In the modern world of medicine, a huge variety of medical equipment has been developed for successfully performing and aiding medical treatments. The medical equipment ranges from large precision detection devices to tiny, easy-to-use medical accessories. The air permeable tape that is used to affixing bandages is one of such examples.

Air permeable tape is widely used in medical treatments, especially in affixing other medical instruments such as injection needle or gauze pad onto human body. Referring to FIG. 1, a perspective view of a conventional air permeable tape is illustrated. The air permeable tape 10 includes a cloth 11 and epoxy layer 12. The cloth 11 can be made of many kinds of soft materials, while the epoxy layer 12 is sprayed or coated on one surface of the cloth 11 for adhering the air permeable tape 10 on human body. However, since the air permeability of the conventional air permeable tape 10 is quite low, a plurality of holes 13 is punched on the tape 10 to enhance the air permeability. In addition to the spraying or coating method used for applying the epoxy layer 12 on the cloth 11, the epoxy layer 12 can also be formed by using pressure sensitive thermal glue. The thermal glue is transferred to the cloth 11 by means of a pressure roller, or permeated to the fiber of the cloth 11 to achieve similar adhesive effect. The sectional view of the holes 13, cloth 11 and epoxy layer 12 of a conventional air permeable tape is illustrated in FIG. 1A.

### SUMMARY OF THE INVENTION

Since in the conventional air permeable tape, a layer of pressure sensitive thermal glue that is not air permeable is used, the thermal glue will cover the holes formed in the cloth fiber when combining the epoxy layer and the cloth, which worsens the air permeability. Therefore, a plurality of holes is punched on the conventional air permeable tapes to improve the air permeability.

However, punching holes on the air permeable tapes requires additional manufacturing processes. On the other hand, since there are still a significant portion of the conventional air permeable tapes not being punched a hole, punching holes on the air permeable tape can not solve the air permeability problem completely. If such tapes are used for a long time, the water vapor (e.g. sweat, etc.) generated from human skin will induce the growth of bacteria, which can easily render a wound being infected. In addition, after the conventional air permeable tape being rinsed in the sweat generated from human skin, the epoxy layer is easily disseminated from the cloth. Therefore, some epoxy layer will remain on the skin when tearing the tape off, which is rather inconvenient.

In light of the above, one objective of the present invention is to provide an air permeable tape that can solve the air permeability problem mentioned above, as well as reducing the manufacturing process. The air permeable tape of the present invention can be affixed to any place of a human body. Since a plurality of air permeable holes is formed on the air permeable tape, the water vapor generated from human skin is sufficiently dissipated to the air. In addition, a layer of adhesive is formed on the fiber surface, the air permeable tape can easily be affixed and tore off from the skin.

Hereinafter, the air permeable tape of the present invention is described in detail by incorporating with the accompanying drawings.

### BRIEF DESCRIPTION OF THE DRAWINGS

FIG. 1 is a perspective view of a conventional air permeable tape.

FIG. 1A is a sectional view of the conventional air permeable tape.

FIG. 2 is a perspective view of an air permeable tape in accordance with one embodiment of the present invention.

FIG. 2A is a detailed perspective view of the air permeable tape of the present invention.

FIG. 3 is a perspective view of the air permeable tape in accordance with another embodiment of the present invention.

FIG. 3A is a sectional view of the air permeable tape of the present invention.

FIG. 4 illustrates the usage of the air permeable tape of the present invention.

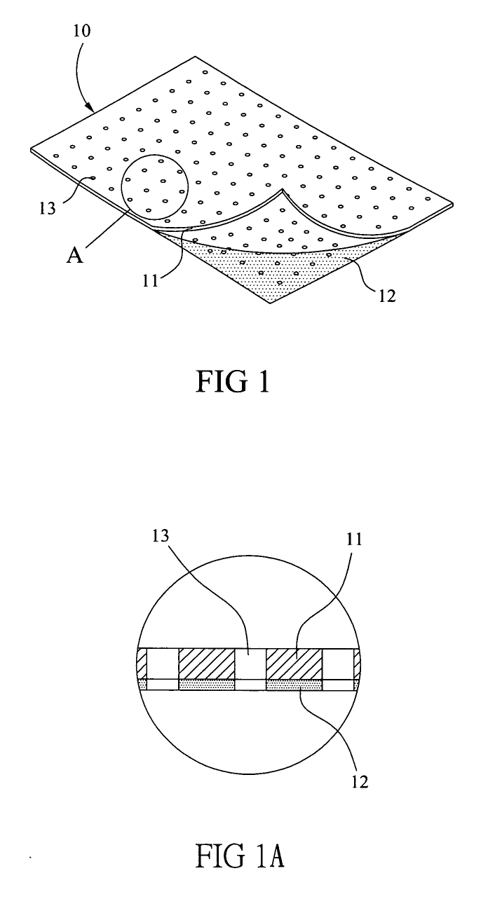

### DETAILED DESCRIPTION OF THE PREFERED EMBODIMENTS

The novel features of the present invention are hereinafter described in detail by incorporating with the accompanying drawings. However, it is appreciated that the accompanied drawings are provided only for the convenience of illustration and description, no limitation is intended thereto.

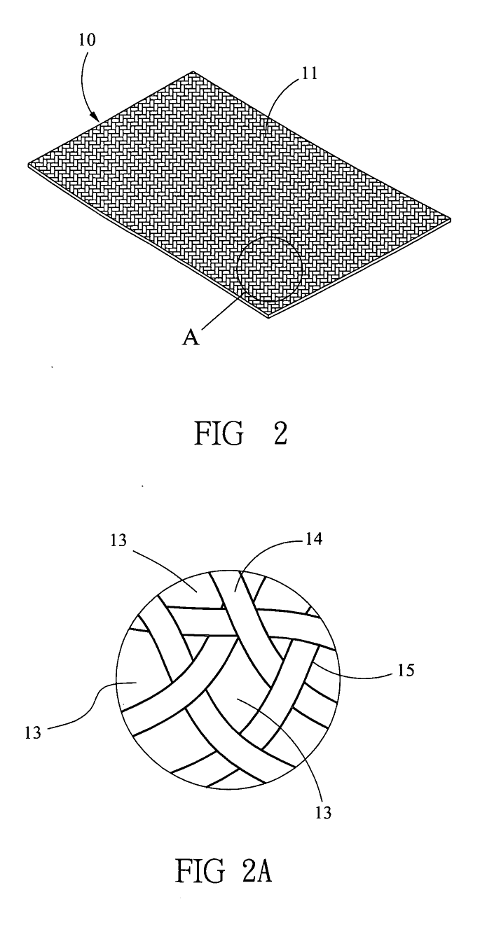

Referring to FIG. 2, a perspective view of the air permeable tape in accordance with one embodiment of the present invention is illustrated. The air permeable tape 10 includes a cloth 11. The cloth 11 is made of fibrous polymer materials. The fibrous polymer material includes non-woven fabrics, nylon cotton or other fibrous cloth. Referring to FIG. 2A, a detailed perspective view of the air permeable tape of the present invention is illustrated. The cloth 11 is composed of a plurality of interwovenly constructed fibers 14. The fibers 14 are irregularly integrated and arranged in an arbitrary manner. Since fibers 14 are arranged irregularly, air permeable holes 13 are formed between the fibers 14. In addition, a layer of adhesive 15 is formed on the surface of each piece of fiber 14. The adhesive 15 on the fiber 14 of the cloth 11 provide the means for securely affixing the air permeable tape 10 onto the skin of the user.

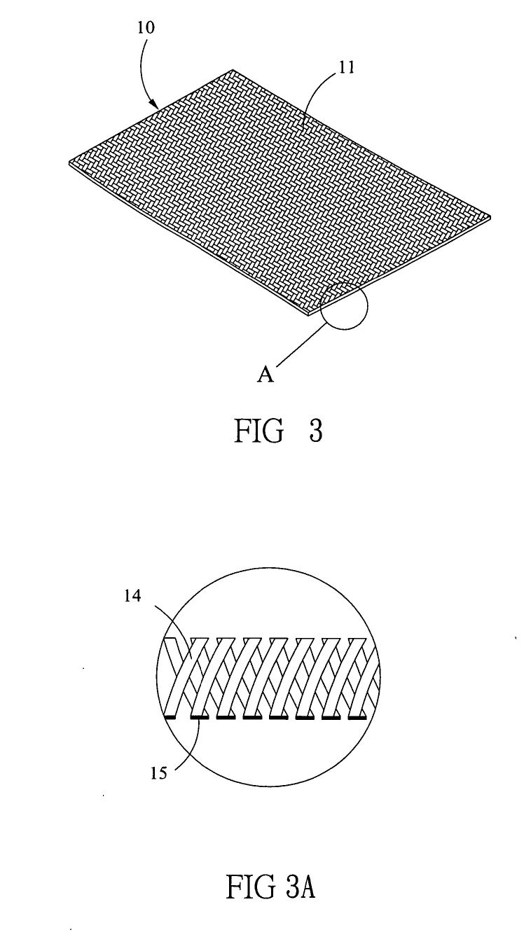

Referring to FIG. 3, a perspective view of the air permeable tape in accordance with another embodiment of the present invention is illustrated. The air permeable tape 10 includes a cloth 11. The cloth 11 is made of fibrous polymer materials. The fibrous polymer material includes non-woven fabrics, nylon cotton or other fibrous cloth. Referring to FIG. 3A, a sectional view of the air permeable tape of the present invention is illustrated. The cloth 11 is composed of a plurality of interwovenly constructed fibers 14. The fibers 14 are irregularly integrated and arranged in an arbitrary manner. Since fibers 14 are arranged irregularly, air permeable holes 13 are formed between the fibers 14. As shown, the surface of some fibers 14 forms the contact surface of the cloth 11 that contacts the skin of the user. Since a layer of adhesive 15 is formed on the surface of each piece of fiber 14, the adhesive 15 on the fiber 14 of the cloth 11 provide the means for securely affixing the air permeable tape 10 onto the skin of the user.

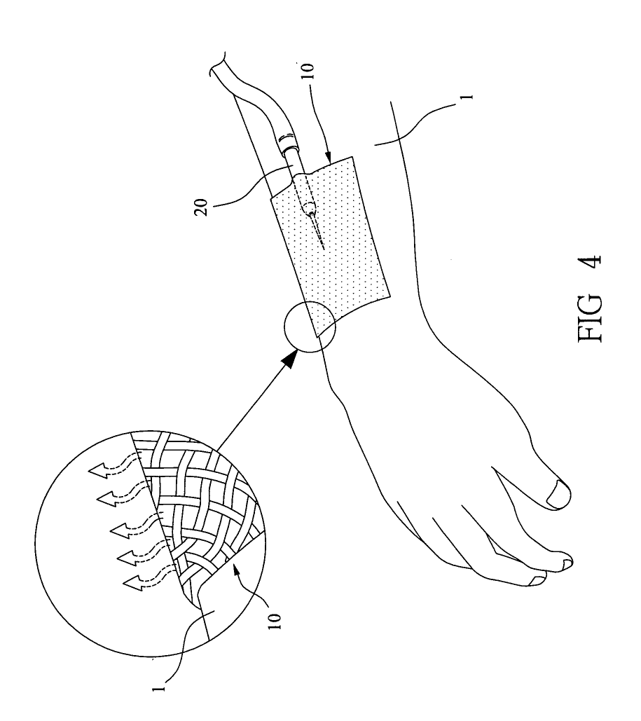

Referring to FIG. 4, one usage example of the air permeable tape of the present invention is illustrated. As shown, the air permeable tape 10 is used to affix the injection needle 20 on the skin surface. Since the air permeable tape 10 includes a huge amount of air permeable holes, the water vapor generated from the skin surface 1 can be dissipated to the air through the air permeable holes. Since the conventional air permeable tape does not have air permeable holes, the water vapor generated from the skin surface can not be dissipated to the air, thereby resulting in [symbol] . In addition, since only the adhesive on the fibers is used to contact with human skin, the employment of adhesives can largely be reduced. In this manner, the production cost is reduced and the tape is easier to tear down as well.

Since, any person having ordinary skill in the art may readily find various equivalent alterations or modifications in light of the features as disclosed above, it is appreciated that the scope of the present invention is defined in the following claims. Therefore, all such equivalent alterations or modifications without departing from the subject matter as set forth in the following claims is considered within the spirit and scope of the present invention.

What is claimed is:

1. An air permeable tape for adhering on human skin, comprising a cloth being composed of a plurality of fibers, the fibers forming a plurality of air permeable holes, and the surface of each fiber being coated with an adhesive, whereby the adhesive contacts the human skin and securely affixes the air permeable tape on the human skin.

2. The air permeable tape as recited in claim 1, wherein the cloth is made of fibrous polymer material.

3. The air permeable tape as recited in claim 2, wherein the fibrous polymer material comprises non-woven fabrics.

4. An air permeable tape for adhering on human skin, comprising a cloth being composed of a plurality of fibers, the fibers forming a plurality of air permeable holes, and the surface of fiber near the surface of the cloth being coated with an adhesive, whereby the adhesive contacts the human skin and securely affixes the air permeable tape on the human skin.

5. The air permeable tape as recited in claim 1, wherein the cloth is made of fibrous polymer material.

6. The air permeable tape as recited in claim 2, wherein the fibrous polymer material comprises non-woven fabrics.
